(19) United States
(12) Patent Application Publication
(10) Pub. No.: US 2026/0356797 A1
(43) Pub. Date: Nov. 12, 2026

(54) METHOD FOR MANUFACTURING A SOLUTION OF BIS(FLUORO SULFONYL)IMIDE SALTS

(71) Applicant: SPECIALTY OPERATIONS FRANCE, Lyon (FR)

(72) Inventors: Etienne Schmitt, Soucieu-en-Jarrest (FR); Richard Smith, Sainte-Foy-lès-Lyon (FR)

(73) Assignee: SPECIALTY OPERATIONS FRANCE, Lyon (FR)

(21) Appl. No.: 19/139,835

(22) PCT Filed: Nov. 21, 2023
(86) PCT No.: PCT/EP2023/082567

§ 371 Date: Jun. 16, 2025

(30) Foreign Application Priority Data
Dec. 15, 2022 (EP) 22306895.8

(51) Int. Cl. C07C 303/36; C07C 303/44

(52) U.S. Cl. CPC C07C 303/36; C07C 303/44

(57) ABSTRACT

A method for manufacturing a composition comprising a salt of bis(fluoro sulfonyl)imide (salt-FSI), the method comprising the steps of: providing a composition [composition (COMP)] comprising: at least a salt of bis(fluoro sulfonyl)imide [salt-FSI] in a first concentration, and at least one solvent [solvent (S)]; charging said composition (COMP) into a first vessel; charging at least one organic solvent [solvent (S1)] into the first vessel; subjecting said composition (COMP) and the solvent (S1) to a first distillation; recovering a composition [composition (COMP-F)] in the form of a solution comprising: the at least one salt-FSI in a second concentration wherein the second concentration is higher than the first concentration in the composition (COMP), water in an amount lower than the amount of water in composition (COMP), and at least one solvent comprising said solvent (S1) in an amount of at least 95 wt. % based on the total weight of the composition (COMP-F).

### CROSS-REFERENCE TO RELATED PATENT APPLICATIONS

This application claims priority filed on 15 Dec. 2022 in Europe with Nr. 22306895.8, the whole content of this application being incorporated herein by reference for all purposes.

### TECHNICAL FIELD

The present invention relates to a method for the manufacture of a solution containing bis(fluoro sulfonyl)imide salt(s).

### BACKGROUND

Bis(fluoro sulfonyl)imide (FSI) and salts thereof, in particular the lithium salt of bis(fluoro sulfonyl)imide (LiFSI), are useful compounds in a variety of technical fields, including for the manufacture of battery electrolytes.

Several methods for the manufacture of FSI and its salts have been described in the art. Among the various technologies described, most of the manufacturing methods comprise a fluorination reaction, wherein a fluorinating agent is reacted with a suitable compound, in a solvent.

Many efforts have been taken in the art to improve the manufacturing methods of FSI salts and of the intermediate compounds thereof, in particular with regard to purity and yield of the intermediate and final compounds and cost reduction of the overall manufacturing method. Also, salts of FSI are known to be sensitive to water and can react to form unwanted species, which severely degrade the quality and the electrochemical properties of the FSI product.

For example, US 2019/0292053 (Arkema France) discloses a method for drying and purifying a lithium bis(fluoro sulfonyl)imide salt in solution in an organic solvent S1, which comprises the steps of: a) adding deionised water to dissolve and extract the lithium bis(fluoro sulfonyl)imide salt [LiFSI], forming an aqueous solution of said salt; b) extracting the LiFSI from said aqueous solution, using an organic solvent S2; c) concentrating the LiFSI by evaporating said organic solvent S2and the water, in a short-path thin-film evaporator; and d) optionally, crystallizing the LiFSI.

### SUMMARY OF THE INVENTION

The Applicant is aware that despite all the attempts in the art, there is still the need for a method for the manufacture of a solution containing bis(fluoro sulfonyl)imide salt(s) (salt-FSI), which is efficient and can be easily scaled up from laboratory to pilot and industrial scale.

The Applicant is also aware that FSI salts are very sensitive to water. Hence, the Applicant faced the problem of developing a method that avoids contacting a raw salt-FSI salt with a large amount of water.

More in particular, the Applicant faced the problem of providing a method, which allows to manufacture a solution of bis(fluoro sulfonyl)imide salt(s) (salt-FSI) with no degradation of the same and a high recovery yield, even when applied at industrial scale.

Surprisingly, the Applicant developed a new method for the manufacture of a salt-FSI, which meets the above mentioned criteria and needs and that can be easily scaled up from the laboratory scale to the industrial plant.

An advantage of the method of the present invention is that a solution of a salt of bis(fluoro sulfonyl)imide is obtained, which is characterized by a very high purity and low water content, and no degradation of the starting material occurs.

Another advantage of the method of the present invention is that the organic solvent(s) and the water obtained as waste products can be further recycled.

### DISCLOSURE OF THE INVENTION

In the present application:
the numerical ranges includes the limits, unless otherwise specified;
any description, even though described in relation to a specific embodiment, is applicable to and interchangeable with other embodiments of the present invention;
where an element or component is said to be included in and/or selected from a list of recited elements or components, it should be understood that in related embodiments explicitly contemplated here, the element or component can also be any one of the individual recited elements or components, or can also be selected from a group consisting of any two or more of the explicitly listed element components may be omitted from such list;
the term “ppm” (or “part per million”) refers to a weight fraction over the total weight of the composition, unless specified otherwise.

The present invention relates to a method for manufacturing a composition comprising a salt of bis(fluoro sulfonyl)imide (salt-FSI), said composition being in the form of a solution and said method comprising the steps of:
(I) providing a composition [composition (COMP)] comprising:
   at least a salt of bis(fluoro sulfonyl)imide [salt-FSI] in a first concentration (salt-FSI 1), and
   at least one solvent [solvent (S)], wherein said solvent(S) comprises water in an amount of at least 1 wt. % based on the overall weight of said solvent (S);
(II) charging said composition (COMP) into a first vessel;
(III) charging at least one organic solvent [solvent (S1)] into said first vessel;
(IV) subjecting said composition (COMP) and said solvent (S1) to a first distillation;
(V) recovering a composition [composition (COMP-F)] in the form of a solution comprising:
   said at least one salt-FSI in a second concentration (salt-FSI 2), said second concentration (salt-FSI 2) being higher than said first concentration (salt-FSI 1) in composition (COMP),
   water in an amount lower than the amount of water in composition (COMP), and
   at least one solvent [solvent (S*)] comprising said solvent (S1) in an amount of at least 95 wt. % based on the total weight of composition (COMP-F).

Preferably, said salt-FSI is a salt of bis(fluoro sulfonyl)imide with one of: lithium, sodium, potassium, zinc, or magnesium. Lithium, sodium and potassium are more preferred.

The term “injecting” or “inject” or “injection” hereby means that the composition described herein is placed into a vessel (also called recipient or device) suitable for performing the subsequent step of distillation. Within the context of the present invention, “injecting the composition in the vessel” is equivalent to “adding the composition to the vessel” or to “feeding the composition to the vessel”.

The term “recovering” hereby means that the composition (COMP-F) is removed or extracted or withdrawn from the vessel wherein the previous step(s) take(s) place.

The term “vessel” hereby means a container which is well-suited for the method of the present invention, that-is-to-say which is adapted to withstand the pressures and temperatures used in the method of the present invention, as well as to the possible corrosive character of the reactants and products involved in this method.

The method of the present invention can be implemented in batch, semi-continuous or continuous mode.

The configuration of the equipment for performing the method of the present invention is not limited.

Preferably, for performing the method of the present invention, such a vessel is preferably a distillation column, or a container, such as a boiler, equipped with a distillation column.

According to a preferred embodiment, in step (I), composition (COMP) comprises at least a salt-FSI in a first concentration (salt-FSI 1) and at least a solvent(S) comprising water in an amount of 100 wt. % based on the weight of solvent(S). According to this embodiment, solvent(S) consists of water. Also, according to this embodiment, solvent(S) comprises no organic solvent.

Preferably, said composition (COMP) comprises said salt-FSI in a concentration from about 0.1 to about 35 wt. %, more preferably from about 0.5 to 30 wt. % and even more preferably from 1 to 20 wt. % based on the total weight of the composition (COMP).

Alternatively, said solvent(S) comprises water in an amount of at least 5.0, 7.0, 12.0, 17.0, 22.0, 27.0, 32.0, 37.0, 42.0, 47.0, 52.0, 57.0, 62.0, 67.0, 72.0, 77.0, 82.0, 87.0, 92.0, 97.0, 98.0, 99.9 wt. % based on the weight of solvent(S).

For example, the amount of water is from about 65.0 to about 99.9 wt. %, more preferably from about 70.0 to about 99.5 wt. % and even more preferably from 80.0 to 99.0 wt. % based on the total weight of solvent(S).

When the amount of water in composition (COMP) is different from 100 wt. % based on the total weight of solvent(S), said solvent(S) comprises at least one organic solvent. For example, such at least one organic solvent is at least one solvent that is miscible with water. As an example, such organic solvent is selected from alcohols.

The method for manufacturing composition (COMP) is not limited.

Composition (COMP) can be prepared according to any method known in the art. For example, a predetermined amount of salt-FSI in the solid form is dissolved into a suitable solvent(S) as defined above. Said solvent is preferably solvent(S) as defined above, and more preferably solvent(S) consists of water.

Preferably, step (II) is performed via a pump or by gravity.

In step (II), composition (COMP) is charged into the vessel via a proper mean. Preferably, such a proper mean is a nozzle or an injector.

In step (II), composition (COMP) can be charged sequentially, either semi-continuously or continuously.

Preferably, composition (COMP) is continuously charged into said first vessel.

Alternatively, composition (COMP) is semi-continuously charged into the vessel. For example, composition (COMP) is charged into the vessel for a certain time, such as for example between 30 and 120 seconds, preferably for about 60 seconds, and then the charging is stopped for another period of time, which can be equal to, shorter or longer than the charging time.

Preferably, in step (III), said at least one solvent (S1) is selected in the group comprising optionally fluorinated carbonate solvents, such as: ethylene carbonate, fluoroethylene carbonate, vinylene carbonate, propylene carbonate, butylene carbonate, dimethyl carbonate, ethyl methyl carbonate, diethyl carbonate; lactones, such as: γ-butyrolactone, γ-valerolactone; ethers, such as: dimethoxymethane, 1,2-dimethoxyethane, tetrahydrofuran, 2-methyltetrahydrofuran, 1,3-dioxane, 4-methyl-1,3-dioxolane; optionally fluorinated esters, such as: methyl formate, methyl acetate, ethyl acetate, methyl propionate, isopropyl acetate, n-butyl acetate, n-propyl propionate, 2,2-difluoroethyl acetate; polar aprotic solvents, such as sulfolane, 3-methyl sulfolane, dimethylsulfoxide, N, N-dimethylformamide, N-methyl oxazolidinone, acetonitrile, valeronitrile, benzonitrile, nitromethane and nitrobenzene.

More preferably, said solvent (S1) is selected from optionally fluorinated carbonate solvents and esters, as listed above.

Even more preferred solvent (S1) is selected from dimethyl carbonate, ethyl methyl carbonate, diethyl carbonate, ethyl acetate, isopropyl acetate and n-butyl acetate.

According to a more preferred embodiment, said solvent (S1) is selected from ethyl methyl carbonate (EMC), dimethyl carbonate and n-butyl acetate.

Preferably, step (III) is performed via a pump or by gravity.

Preferably, said step (III) is performed by charging solvent (S1) sequentially, either semi-continuously or continuously.

Preferably, step (II) and step (III) are performed simultaneously.

Alternatively, step (III) is started after step (II) or when step (II) is finished or stopped.

Preferably, step (IV) is performed via a distillation column.

According to an embodiment, step (IV) is started after each of step (II) and step (III) is finished or stopped.

Alternatively, step (IV) is started while step (II) and/or step (III) are ongoing.

Preferably, step (IV) is performed at a pressure below 50 mbar abs (0.005 MPa), more preferably below 40 mbar abs (0.004 MPa). According to a preferred embodiment, step (IV) is performed at a pressure between 25 and 40 mbar abs (0.0025 and 0.004 MPa).

Preferably, step (IV) is performed at a temperature lower than 50° C., more preferably lower than 40° C. According to a preferred embodiment, step (IV) is performed at a temperature between 20 and 35° C.

The method of the present invention optionally comprises after step (IV) and before step (V), a step (IV-a) of charging the composition obtained in step (IV) into a second vessel.

Optionally, the method according to the present invention comprises after step (IV) or after step (IV-a), step (IV-b) of charging at least one second organic solvent [solvent (S2)] into said first vessel or into said second vessel and step (IV-c) of performing a second distillation.

In step (IV-b), said solvent (S2) is the same or different from said solvent (S1).

Preferably, solvent (S2) is selected from the group comprising the list of solvents disclosed above for solvent (S1).

According to a preferred embodiment, said solvent (S2) is the same as solvent (S1).

Preferably, step (VI-c) is performed at a pressure lower than the pressure applied in step (IV). Preferably, step (IV-c) is performed at pressure lower than 35 mbar abs (0.0035 MPa), even more preferably lower than 25 mbar abs (0.0025 MPa).

Preferably, step (IV-c) is performed at a temperature lower than 50° C., more preferably lower than 40° C. According to a preferred embodiment, step (IV) is performed at a temperature between 20 and 35° C.

According to a preferred embodiment, composition (COMP-F) in step (V) comprises said at least one salt-FSI in a second concentration (salt-FSI 2), said second concentration (salt-FSI 2) being higher than said first concentration (salt-FSI 1) in composition (COMP), water in an amount lower than 30 000 ppm based on the weight of said salt-FSI and solvent (S*) comprising solvent (S1) in an amount higher than 97.0 wt. % based on the total weight of solvent (S*).

Preferably, said composition (COMP-F), comprises an salt-FSI in an amount of from about 0.2 to about 70 wt. %, more preferably from about 1.0 to 60 wt. % and even more preferably from 2 to 40 wt. % based on the total weight of the composition (COMP-F).

Preferably, said composition (COMP-F) comprises water in an amount below 10000 ppm, preferably below 1000 ppm, more preferably below 500 ppm and even more preferably below 100 ppm, based on the total weight of said salt-FSI.

According to a more preferred embodiment, said composition (COMP-F) comprises water in an amount below 80 ppm, more preferably below 50 ppm and even more preferably below 20 ppm, based on the total weight of said salt-FSI.

Preferably, solvent (S*) comprises solvent (S1), optionally in admixture with solvent (S2), in an amount higher than 98.0 wt. %, more preferably higher than 99.0 wt. % and even more preferably higher than 99.5 wt. %.

More preferably, solvent (S*) comprises solvent (S1), optionally in admixture with solvent (S2), in an amount higher than 99.7 wt. %, the amounts being based on the total weight of solvent (S*). Even more preferably, solvent (S*) consists of solvent (S1).

Preferably, composition (COMP-F) contains at least one other substance.

Said at least one other substance is preferably selected from:
fluoride (F⁻) preferably in an amount less than 100 ppm, as measured by lonic Chromatography (IC); and/or
chloride (CI⁻) preferably in an amount less than 100 ppm, as measured by IC; and/or
sulfate (SO₄²⁻) preferably in an amount less than 1000 ppm, as measured by IC; and/or
sulfamate (NH₂SO₃⁻) preferably in an amount less than 1000 ppm, as measured by IC; and/or
fluorosulfonate (FSO₃⁻) preferably in an amount less than 1000 ppm, as measured for example by lonic Chromatography (IC), the amounts being based on the weight of salt-FSI.

Preferably, the amounts of said at least one other substance in composition (COMP-F) are preferably as follows:
fluoride (F⁻) in an amount up to 50 ppm, for example less than 40 ppm, less than 30 ppm, less than 25 ppm;
chloride (Cl⁻) in an amount up to 50 ppm, for example less than 40 ppm, less than 30 ppm, less than 20 ppm, less than 10 ppm or even less than 8 ppm;
salts different from sulfate (SO₄²⁻) in an amount up to 100 ppm, for example less than 50 ppm, less than 30 ppm, less than 25 ppm; the amounts being measured for example by lonic Chromatography (IC) and being based on the weight of salt-FSI.

Preferably, said salts different from sulfate (SO₄²⁻) are selected from NH₂SO₃⁻ and/or FSO₃⁻.

Preferably, said salts different from sulfate (SO₄²⁻) are in the following amounts:
less than 50 ppm of sulfamate (NH₂SO₃₋), for example less than 40 ppm, less than 30 ppm, less than 20 ppm, less than 10 ppm or even less than 5 ppm as measured by IC; and/or
less than 50 ppm of fluorosulfonate (FSO₃⁻), for example less than 40 ppm, less than 30 ppm, less than 20 ppm, less than 10 ppm or even less than 5 ppm as measured by IC,
the amounts being based on the weight of salt-FSI.

The method of the present invention can further comprise after step (V), a step (V-a) of filter the composition (COMP-F). Such step of filtration can be performed according to methods known in the art. If required by circumstances, a filtration step can be also performed after step (IV) and before step (V) or while step (V) proceeds.

Alternatively or in addition, the method of the present invention can further comprise a step (VI) of concentrating composition (COMP-F). Such step of concentration can be performed according to methods known in the art.

Should the disclosure of any patents, patent applications, and publications which are incorporated herein by reference conflict with the description of the present application to the extent that it may render a term unclear, the present description shall take precedence.

The present invention will be now described in more detail with reference to the following examples, whose purpose is merely illustrative and not intended to limit the scope of the disclosure.

### EXAMPLES

### Example 1

Into a 2.5 L PFA-coated vessel with 4 baffles, equipped with a mechanically stirred 4-blades PTFE shaft, a Raschig-garnished 30 cm distillation column, a glass distillation head and a Bertrand separator, and connected to a cold trap and a vacuum pump, high purity lithium bis(fluorosulfonyl)imide salt (10.0 g) was dissolved with distilled water (90.3 g) and loaded into the vessel. Ethyl methyl carbonate (EMC) (902.4 g) was loaded to the same vessel. The mixture was stirred at 300 rpm and the distillation was started.

A first azeotropic distillation step was performed at 30-35 mbar abs (0.0030-0.0035 MPa) and 25-30° C. (boiler's temperature), allowing the distillation of a biphasic mixture. A sample of the resulting LiFSI solution (Sample 1:538.3 g) was analyzed by Karl-Fisher titration (KF) and ionic chromatography (IC).

Fresh EMC was added (670 g) and a second azeotropic distillation step was performed at 8-20 mbar abs (0.0008-0.0020 MPa) and 29-31° C. (boiler's temperature) providing a final LiFSI solution in EMC. This composition (Sample 2) was analyzed by KF, NMR, and IC.

### KF Analysis

Sample 1: 2.3 wt. % LiFSI in EMC, with a water content of about 13900 ppm to the weight percent of LiFSI.

Sample 2: 45 wt% LiFSI in EMC, with a water content of less than 2500 ppm to the weight percentage of LiFSI.

IC analysis showed that no chemical degradation occurred while performing the process.

All the above results showed that the method according to the present invention allowed the preparation of a solution of a salt of FSI in an organic solvent, while the water and solvent removed via distillation can be recycled in the same or in a different process.

1. A method for manufacturing a composition comprising a salt of bis(fluoro sulfonyl)imide (salt-FSI), said composition being in a form of a solution and said method comprising the steps of:
(I) providing a composition [composition (COMP)] comprising:
at least a salt of bis(fluoro sulfonyl)imide [salt-FSI] in a first concentration (salt-FSI 1), and
at least one solvent [solvent (S)], wherein said solvent(S) comprises water in an amount of at least 1 wt. % based on [[the]] an overall weight of said solvent (S);
(II) charging said composition (COMP) into a first vessel;
(III) charging at least one organic solvent [solvent (S1)] into said first vessel;
(IV) subjecting said composition (COMP) and said solvent (S1) to a first distillation;
(V) recovering a composition [composition (COMP-F)] in a form of a solution comprising:
said at least one salt-FSI in a second concentration (salt-FSI 2), said second concentration (salt-FSI 2) being higher than said first concentration (salt-FSI 1) in the composition (COMP),
water in an amount lower than the amount of water in the composition (COMP), and
at least one solvent [solvent (S*)] comprising said solvent (S1) in an amount of at least 95 wt. % based on a total weight of the composition (COMP-F).

2. The method according to claim 1, wherein said salt-FSI is a salt of bis(fluoro sulfonyl)imide with one of: lithium, sodium, potassium, zinc, or magnesium.

3. The method according to claim 1, wherein in step (I), the composition (COMP) comprises at least the salt-FSI in the first concentration (salt-FSI 1) and at least the solvent(S) comprising water in an amount of 100 wt. % based on the weight of the solvent(S).

4. The method according to claim 3, wherein the solvent(S) comprises no organic solvent.

5. The method according to claim 1, wherein said composition (COMP) comprises said salt-FSI in an amount from about 0.1 to about 35 wt. %, based on the total weight of the composition (COMP).

6. The method according to claim 1, wherein said at least one solvent (S1) is selected in the group comprising optionally fluorinated carbonate solvents.

7. The method according to claim 1, wherein step (II) and step (III) are performed simultaneously.

8. The method according to claim 1, wherein step (IV) is performed:
via a distillation column; and/or
at a pressure below 50 mbar abs (0.005 MPa); and/or
at a temperature lower than 50° C.

9. The method according to claim 1, said method comprising after step (IV) and before step (V), a step (IV-a) of charging the composition obtained in step (IV) into a second vessel.

10. The method according to claim 9, wherein the method comprises after step (IV) or after step (IV-a), the following steps:
(IV-b) charging at least one second organic solvent [solvent (S2)] into said first vessel or into said second vessel and
(IV-c) performing a second distillation.

11. The method according to claim 10, wherein said solvent (S2) is selected in the group comprising optionally fluorinated carbonate solvents, lactones, optionally fluorinated esters, polar aprotic solvents.

12. The method according to claim 10, wherein:
the solvent (S2) is the same as the solvent (S1); and/or
step (IV-c) is performed at a pressure lower than the pressure applied in step (IV).

13. The method according to claim 1, wherein the composition (COMP-F) comprises:
said at least one salt-FSI in a second concentration (salt-FSI 2), said second concentration (salt-FSI 2) being higher than said first concentration (salt-FSI 1) in the composition (COMP),
water in an amount lower than 30 000 ppm based on the weight of said salt-FSI and
the solvent (S*) comprising the solvent (S1) in an amount higher than 97.0 wt. % based on a total weight of solvent (S*).

14. The method according to claim 10, wherein the composition (COMP-F) further comprises:
one or both of said solvent (S1) and said solvent (S2), in an amount lower than 100 ppm based on the total weight of the composition (COMP-F); and/or
at least one other substance selected from:
fluoride (F⁻) in an amount less than 100 ppm, as measured by Ionic Chromatography (IC); and/or
chloride (Cl⁻) in an amount less than 100 ppm, as measured by IC; and/or
sulfate (SO₄²⁻) in an amount less than 1000 ppm, as measured by IC; and/or
sulfamate (NH₂SO₃⁻) in an amount less than 1000 ppm, as measured by IC; and/or
fluorosulfonate (FSO₃⁻) in an amount less than 1000 ppm, as measured for example by Ionic Chromatography (IC),
the amounts being based on the weight of the salt-FSI.

15. The method according to claim 1, said method comprising, after step (V):
a step (V-a) of filtering the composition (COMP-F); and/or
a step (VI) of concentrating the composition (COMP-F).

16. The method according to claim 1, wherein said composition (COMP) comprises said salt-FSI in an amount from about 0.5 to 30 wt. % based on the total weight of the composition (COMP).

17. The method according to claim 6, wherein the fluorinated carbonate solvents are selected from the group of: ethylene carbonate, fluoroethylene carbonate, vinylene carbonate, propylene carbonate, butylene carbonate, dimethyl carbonate, ethyl methyl carbonate, and diethyl carbonate.

18. The method according to claim 6, wherein the lactones are selected from the group of γ-butyrolactone and γ-valerolactone.

19. The method according to claim 6, wherein the ethers are selected from the group of dimethoxymethane, 1,2-dimethoxyethane, tetrahydrofuran, 2-methyltetrahydrofuran, 1,3-dioxane, and 4-methyl-1,3-dioxolane.

20. The method according to claim 6, wherein the fluorinated esters are selected from the group of methyl formate, methyl acetate, ethyl acetate, methyl propionate, isopropyl acetate, n-butyl acetate, n-propyl propionate, and 2,2-difluoroethyl acetate.
